(19) United States
(12) Patent Application Publication
(10) Pub. No.: US 2022/0370129 A1
(43) Pub. Date: Nov. 24, 2022

(54) SYSTEMS AND METHODS FOR GENERATING A MODULATED LASER PULSE

(71) Applicant: Boston Scientific Scimed, Inc., Maple Grove, MN (US)

(72) Inventors: Baocheng YANG, Fremont, CA (US); Xirong YANG, Fremont, CA (US); Brian CHENG, San Jose, CA (US); Peter BULL, San Jose, CA (US); Rongwei Jason XUAN, Fremont, CA (US); Jian James ZHANG, Lancaster, MA (US); Thomas Charles HASENBERG, Campbell, CA (US); David PIH, Milpitas, CA (US)

(73) Assignee: Boston Scientific Scimed, Inc., Maple Grove, MN (US)

(21) Appl. No.: 17/664,300

(22) Filed: May 20, 2022

Related U.S. Application Data
(60) Provisional application No. 63/191,535, filed on May 21, 2021.

(51) Int. Cl. A61B 18/24; A61B 18/26; A61B 17/22

(52) U.S. Cl. CPC A61B 18/24; A61B 18/26; A61B 17/22004; A61B 2017/00176

(57) ABSTRACT

A medical laser system for outputting laser pulses includes at least one laser cavity, a rotating mirror, a user interface, and a controller. The controller is configured to receive at least one laser parameter associated with a laser pulse output by the system. The controller is configured to determine an average power level of the laser pulse based on the at least one laser parameter associated with the laser pulse. The controller is configured to determine a pulse width modulation (PWM) control signal based on at least one laser parameter. The controller is configured to generate the laser pulse based on the average power level and the PWM control signal, the laser pulse comprising at least one of a first shape, a second shape, or a third shape. Each of the first shape, the second shape, and the third shape of the laser pulse includes different pulse widths.

### CROSS-REFERENCE TO RELATED APPLICATIONS

This application claims the benefit of priority from U.S. Provisional Application No. 63/191,535, filed on May 21, 2021, which is incorporated by reference herein in its entirety.

### TECHNICAL FIELD

The present disclosure generally relates to medical/surgical laser systems, and more particularly, to systems and methods for generating a modulated laser pulse with such systems.

### BACKGROUND

Medical laser systems are used for a variety of surgical procedures. These procedures may include dusting and/or fragmentation of stones in the kidney, the bladder, and/or the ureter. Medical laser systems are also used to create incisions and to ablate and/or coagulate soft tissues, such as, but not limited to, the prostate. A conventional medical laser system can produce laser pulses having various pulse wavelengths and pulse profile widths. The pulse profile width of a laser pulse may affect the way the laser pulse interacts with target features (e.g., tissues) of a subject (e.g., a patient), and the pulse profile width of a laser pulse may be adjusted by varying supply voltage applied to a laser cavity. However, the available range of the supply voltage applied to a laser cavity may be relatively limited, as well as being inconvenient and slow to adjust the supply voltage. Consequently, the maximum average power and the pulse mode of a laser pulse output by the conventional medical laser systems may be relatively limited.

### SUMMARY OF THE DISCLOSURE

Examples of the disclosure relate to, among other things, systems and methods for generating modulated laser pulses, among other aspects. Each of the examples disclosed herein may include one or more of the features described in connection with any of the other disclosed aspects.

In one example, a medical laser system may be provided for outputting laser pulses. The medical laser system may include: at least one laser cavity; a rotating mirror; a user interface; and a controller configured to: receive, from the user interface, at least one laser parameter associated with a laser pulse output by the system; determine an average power level of the laser pulse based on the at least one laser parameter associated with the laser pulse; determine a pulse width modulation (PWM) control signal based on at least one laser parameter; and generate the laser pulse based on the average power level and the PWM control signal, the laser pulse comprising at least one of a first shape, a second shape, or a third shape. Each of the first shape, the second shape, and the third shape of the laser pulse may include different pulse widths.

In other aspects, a medical laser system described herein may include one or more of the following features. The least one laser parameter may include at least pulse mode data, pulse repetition frequency data, or pulse energy data associated with the laser pulse. A pulse width of the second shape may be at least 50% greater than a pulse width of the first shape. A pulse width of the third shape of the laser pulse may be at least 2 times greater than a pulse width of the first shape of the laser pulse. A shape of the laser pulse may be determined based at least on pulse mode data. The average power level of the laser pulse may be determined based on a discrete spectrum matrix. The discrete spectrum matrix may include parameters based at least on pulse energy data of the laser pulse, repetition frequency data of the laser pulse, sub-pulse frequency of a PWM pulse, pulse profile width data of the PWM pulse, or overall pulse width data of the laser pulse. The one or more optical channels may include a holmium-doped yttrium aluminum garnet (Ho:YAG) rod. The at least one laser cavity may include four laser cavities. The at least one laser cavity may include at least two laser cavities. The controller may be further configured to: generate a laser pulse having a repetition frequency of 10 Hertz (Hz) or greater by combining multiple laser pulses generated by the at least two laser cavities. The multiple laser pulses may be combined by synchronizing the rotating mirror with the multiple laser pulses generated by the at least laser cavities. The controller may be further configured to: generate the laser pulse having the third shape by combining multiple PWM control signals; and store one or more discrete spectrum matrices comprising parameters specifying one or more pulse modes of the laser pulse.

In another example, a method of outputting laser pulses of a medical laser system may be provided. The method may include: receiving, from a user interface, at least one laser parameter associated with a laser pulse output by the system; determining an average power level of the laser pulse based on the at least one laser parameter associated with the laser pulse; determining a pulse width modulation (PWM) control signal based on the at least one laser parameter; and generating the laser pulse based on the average power level and the PWM control signal, the laser pulse comprising at least one of a first shape, a second shape, or a third shape. Each of the first shape, the second shape, and the third shape of the laser pulse may include different pulse widths.

In other aspects, a method described herein may include one or more of the following features. The at least one laser parameter may include at least pulse mode data, pulse repetition frequency data, or pulse energy data associated with the laser pulse. A pulse width of the second shape may be at least 50% greater than a pulse width of the first shape. A pulse width of the third shape of the laser pulse may be at least 2 times greater than a pulse width of the first shape of the laser pulse. A shape of the laser pulse may be determined based at least on pulse mode data. The average power level of the laser pulse may be determined based on a discrete spectrum matrix. The discrete spectrum matrix may include parameters based at least on pulse energy data of the laser pulse, repetition frequency data of the laser pulse, sub-pulse frequency of a PWM pulse, pulse profile width data of the PWM pulse, or overall pulse width data of the laser pulse. The medical laser system may generate a laser pulse having a repetition frequency of 10 Hertz (Hz) or greater by combining multiple laser pulses generated by at least two laser cavities. The multiple laser pulses may be combined by synchronizing a rotating mirror with the multiple laser pulses generated by the at least two laser cavities. The medical laser system may generate the laser pulse having the third shape by combining multiple PWM control signals. The medical laser system may store one or more discrete spectrum matrices comprising parameters specifying one or more pulse modes of the laser pulse.

In yet another example, a non-transitory computer-readable medium may store instructions for outputting laser pulses of a medical laser system. The instructions, when executed by one or more processors, may cause the one or more processors to perform operations. The operations may include: receiving, from a user interface, at least one laser parameter associated with a laser pulse output by the system; determining an average power level of the laser pulse based on the at least one laser parameter associated with the laser pulse; determining a pulse width modulation (PWM) control signal based on the at least one laser parameter; and generating the laser pulse based on the average power level and the PWM control signal, the laser pulse comprising at least one of a first shape, a second shape, or a third shape. Each of the first shape, the second shape, and the third shape of the laser pulse may include different pulse widths.

### BRIEF DESCRIPTION OF THE DRAWINGS

The accompanying drawings, which are incorporated in and constitute a part of this specification, illustrate various exemplary embodiments and, together with the description, serve to explain the principles of the disclosed embodiments.

FIG. 1 is a schematic of a medical laser system, according to aspects of this disclosure.

FIG. 2 illustrates an exemplary process of generating laser pulses using the medical laser system of FIG. 1, according to aspects of this disclosure.

FIG. 3 illustrates another exemplary process of generating laser pulses using the medical laser system of FIG. 1, according to aspects of this disclosure.

FIG. 4 illustrates yet another exemplary process of generating laser pulses using the medical laser system of FIG. 1, according to aspects of this disclosure.

FIG. 5 illustrates a flow chart depicting an exemplary method of generating laser pulses using the medical laser system of FIG. 1, according to aspects of this disclosure.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

For ease of description, portions of the disclosed devices and/or their components are referred to as proximal and distal portions. It should be noted that the term “proximal” is intended to refer to portions closer to a laser cavity of the laser system, and the term “distal” is used herein to refer to portions further away from the laser cavity of the laser system, e.g., toward an end of a laser fiber that outputs laser energy. Similarly, extends “distally” indicates that a component extends in a distal direction, and extends “proximally” indicates that a component extends in a proximal direction. Additionally, terms that indicate the geometric shape of a component/surface refer to exact and approximate shapes.

Examples of the disclosure may be used to generate and output laser pulses having one or more pulse modes (or shapes) to one or more treatment sites of a subject (e.g., a patient) using one or more laser cavities of a medical laser system. In some embodiments, the medical laser system may include a controller that may be configured to generate laser pulses having various pulse modes. The controller may receive, from a user interface, at least pulse mode data, pulse repetition frequency data, and pulse energy data associated with a laser pulse output by the system. Further, the controller may determine an average power level of the laser pulse based on the repetition frequency data and the pulse energy data associated with the laser pulse. In embodiments, the controller may determine a pulse width modulation (PWM) control signal based on the pulse mode data. The controller may then generate the laser pulse based on the average power level and the PWM control signal. The laser pulse may have at least one of a first shape, a second shape, or a third shape. Each of the first shape, the second shape, and the third shape of the laser pulse may have different pulse widths.

Examples of the disclosure may relate to systems, devices, and methods for performing various medical procedures and/or treating target features, such as tissues of a subject (e.g., a patient). Reference will now be made in detail to examples of the disclosure described above and illustrated in the accompanying drawings. Wherever possible, the same reference numbers will be used throughout the drawings to refer to the same or like parts.

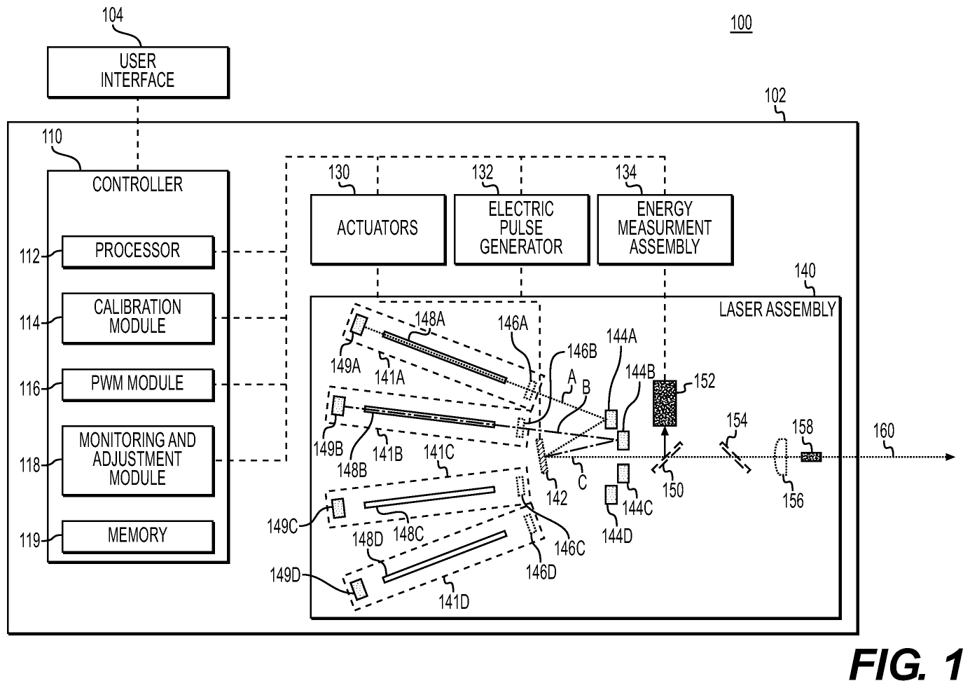

FIG. 1 shows a schematic depiction of an exemplary medical laser system 100 in accordance with an example of this disclosure. The medical laser system 100 may include a laser chassis 102 and a user interface 104. The laser chassis 102 may include a controller 110, actuators 130, an electric pulse generator 132, an energy measurement assembly 134, and a laser assembly 140. The user interface 104 may be communicatively coupled to the controller 110 by, for example, a wired connection, wireless connection, and the like. It should be appreciated that, in some embodiments, the user interface 104 may be a device integral with the medical laser system 100, and in other embodiments, the user interface 104 may be a remote device in communication (e.g., wireless, wired, etc.) with the medical laser system 100. The user interface 104 may include input and output ports to connect with input and output devices such as keyboards, mice, touchscreens, monitors, displays, etc., to receive user inputs and output messages thereon.

Still referring to FIG. 1, the controller 110 may be communicatively coupled to the laser assembly 140 directly or indirectly via the actuators 130, electric pulse generator 132, and/or the energy measurement assembly 134 by, for example, a wired connection, a wireless connection, and the like. In some examples, the controller 110 may be a computer system incorporating a plurality of hardware components that allow the controller 110 to receive data (e.g., laser input parameter data, laser sensor data, etc.), process information (e.g., calibration logic or algorithm, PWM scheme logic or algorithm, monitoring, and adjustment logic or algorithm, etc.), and/or generate control signals to generate and output laser pulses via the laser assembly 140. Illustrative hardware components of the controller 110 may include at least one processor 112, at least one calibration module 114, at least one pulse width modulation (PWM) module 116, at least one monitoring and adjustment module 118, and at least one memory 119.

The processor 112, the calibration module 114, the PWM module 116, and the monitoring and adjustment module 118 of the controller 110 may each include any computing device capable of executing machine-readable instructions, which may be stored on a non-transitory computer-readable medium, for example, the memory 119. By way of example, the processor 112, the calibration module 114, the PWM module 116, and the monitoring and adjustment module 118 may each include an integrated circuit, a microchip, a computer, a memory, and/or any other computer processing unit operable to perform calculations and logic operations required to execute a program. As described in greater detail herein, processor 112, the calibration module 114, the PWM module 116, and the monitoring and adjustment module 118 may each be configured to perform one or more operations in accordance with the instructions stored on the memory 119. The processor 112, the calibration module 114, the PWM module 116, and the monitoring and adjustment module 118 may be communicatively coupled to the actuators 130, the electric pulse generator 132, and the energy measurement assembly 134 in order to facilitate the generation and output of laser pulses by the laser assembly 140.

Still referring to FIG. 1, the calibration module 114 may include executable instructions or algorithms that allow the medical laser system 100 to, for example, calibrate the laser pulses generated by the laser assembly 140. The PWM module 116 may include executable instructions or algorithms that allow the PWM module 116 to, for example, generate and transmit PWM control signals to the electric pulse generator 132. The monitoring and adjustment module 118 may include executable instructions or algorithms that allow the monitoring and adjustment module 118 to, for example, monitor and adjust laser pulses based on one or more signals received from the energy measurement assembly 134 and a pulse energy sensor 152. The electric pulse generator 132 may generate electric pulses based on one or more signals received from the controller (e.g., signals generated by processor 112, calibration module 114, PWM module 116, monitoring and adjustment module 118, etc.) and transmit the generated electric pulses to one or more laser cavities for generating laser (or optical) pulses.

Still referring to FIG. 1, the laser assembly 140 may include one or more laser cavities 141A-D, each laser cavity being configured to output a laser pulse (or laser beam). Each of the one or more laser cavities 141A-D includes a high reflecting window 149A-D at a proximal end, an output coupler window 146A-D at a distal end, and a chromium thulium holmium-doped YAG (CTH:YAG) laser rod 148A-D disposed between a respective high reflecting window 149A-D and an output coupler window 146A-D. A single laser cavity (e.g., laser cavity 141A, 141B, 141C, or 141D) may produce each laser pulse having a pulse wavelength of, for example, approximately 2 μm, and pulse width in the range of 100 microseconds to a few milliseconds. With a single laser cavity, the laser assembly 140 may operate on a repetition frequency (or rate) of approximately 5 Hertz (Hz) to 20 Hz, and the maximum average power output may be approximately 30 Watts. Since the maximum laser pulse energy capable of being generated by a laser cavity decreases with an increase in the operating repetition frequency of the laser cavity, multiple laser cavities may be utilized to achieve greater average power output at relatively higher repetition frequencies (e.g., approximately 20 Hz to 80 Hz). For example, to ablate tissues and to create a high enough heat to destroy objects, such as kidney stones, it may be necessary to increase the repetition frequency of an output laser pulse by utilizing multiple laser cavities. That is, controller 110 may excite each of the multiple laser cavities 141A-D at different times and may rotate rotating mirror 142 in a synchronized manner to match each laser pulse generated by the one or more laser cavities 141A-D. As such, each laser pulse generated by each laser cavity may be combined to produce an output laser pulse having an overall repetition rate of up to approximately 80 Hertz, yielding maximum average power that may be greater than 100 Watts.

Still referring to FIG. 1, each CTH:YAG laser rod 148A-D may generate a laser pulse for each of the laser cavities 141A-D, which is directed to a corresponding relay mirror 144A-D along a laser path (e.g., a laser path A, B, etc.). Each laser pulse is reflected from a respective one of the relay mirrors 144A-D to the rotating mirror 142 (e.g., a Galvo mirror) along respective laser paths. The rotating mirror 142 may be configured to rotate about an axis based on one or more control signals received, for example, from the actuators 130, to face each of the relay mirrors 144A-D and to receive the laser pulses generated by each laser cavity 141A-D. The rotating mirror 142 may reflect each laser pulse from the laser cavities 141A and 141B along with a laser path C to a beam splitter 150 and a beam combiner 154. In one embodiment, the beam splitter 150 may split the laser pulse received via the rotating mirror 142 and transmit a portion of the laser pulse to a pulse energy sensor 152. The energy measurement assembly 134 may receive the pulse signals detected by the energy sensor 152 and may transmit the received pulse signals to the controller 110 for further processing. The beam combiner 154 may combine the laser pulses received from one or more laser cavities 141A-D via the rotating mirror 142. The beam combiner 154 may have a high transmission characteristic for an output laser beam (e.g., a laser pulse having a wavelength of approximately 2.1 um), and a high reflection characteristic for an aiming beam (e.g., an aiming beam having a wavelength of approximate 0.53 um). As such, the beam combiner 154 may combine the output laser beam with the aiming beam incident from a direction perpendicular to that of the output laser beam. Further, the beam combiner 154 may compensate for the transverse shift of the output laser beam introduced by the beam splitter 150. The combined laser pulses may be passed along the laser path C to a coupling lens 156. The coupling lens 156 may couple the combined laser pulses to an output fiber 158 to be transmitted as an output laser pulse (or pulses) 160 to a delivery location. The coupling lens 156 may be any material suitable for coupling the laser light to output fiber 158, including but not limited to a sapphire. The coupling lens 156 may have a focal length of approximately 19 millimeters but is not limited thereto.

In one exemplary embodiment, a laser pulse from the laser cavity 141A may be reflected from the relay mirror 144A to the rotating mirror 142 along the laser path A. Similarly, a laser pulse from the laser cavity 141B may be reflected from the relay mirror 144B to the rotating mirror 142 along the laser path B. The rotating mirror 142 may synchronously reflect each laser pulse from the laser cavities 141A and 141B along the laser path C to the beam splitter 150 and the beam combiner 154. In this example, the overall repetition frequency of the laser cavities 141A and 141B may be between approximately 10 Hz and 40 Hz. Of course, different combinations of laser cavities may be utilized to achieve a desired laser pulse output at different repetition frequencies (or rates).

Still referring to FIG. 1, the medical laser system 100 of this disclosure may generate output laser pulses having different average power levels. The average power of a laser pulse may be characterized by a repetition frequency and a pulse energy level associated with one or more laser cavities 141A-D. For various medical applications, users (or operators) may preset laser pulse energy, repetition frequency, the number of laser cavities desired to be used, etc. In one embodiment, all available average power output levels for laser pulses may be programmed and stored, for example, in memory 119. A complete spectrum of the available average power output of the system 100 may be provided in one or more discrete spectrum matrices, which may be characterized by pulse energy, overall pulse repetition rates, and average optical power. The following table shows an exemplary spectrum matrix (i.e., Pulse Energy Repetition Frequency (PRF) matrix), highlighting one example of available average power levels for given repetition frequencies and pulse energy levels.

TABLE 1.1
Repetition Frequency (Hz)

| | | 5 | 6 | 8 | 10 | 15 | 20 | 25 | 30 | 35 | 40 | 50 | 60 | 70 | 80 |
|---|---|---|---|---|---|---|---|---|---|---|---|---|---|---|---|
| Pulse | 0.2 | 1 | 1.2 | 1.6 | 2 | 3 | 4 | 5 | 6 | 7 | 8 | 10 | 12 | 14 | 16 |
| Energy | 0.3 | 1.5 | 1.8 | 2.4 | 3 | 4.5 | 6 | 7.5 | 9 | 10.5 | 12 | 15 | 18 | 21 | 24 |
| (J) | 0.4 | 2 | 2.4 | 3.2 | 4 | 6 | 8 | 10 | 12 | 14 | 16 | 20 | 24 | 28 | 32 |
| | 0.5 | 2.5 | 3 | 4 | 5 | 7.5 | 10 | 12.5 | 15 | 17.5 | 20 | 25 | 30 | 35 | 40 |
| | 0.6 | 3 | 3.6 | 4.8 | 6 | 9 | 12 | 15 | 18 | 21 | 24 | 30 | 36 | 42 | |
| | 0.8 | 4 | 4.8 | 6.4 | 8 | 12 | 16 | 20 | 24 | 28 | 32 | 40 | 48 | 56 | |
| | 1.0 | 5 | 6 | 8 | 10 | 15 | 20 | 25 | 30 | 35 | 40 | 50 | 60 | | |
| | 1.2 | 6 | 7.2 | 9.6 | 12 | 18 | 24 | 30 | 36 | 42 | 48 | 60 | 72 | | |
| | 1.5 | 7.5 | 9 | 12 | 15 | 22.5 | 30 | 37.5 | 45 | 52.5 | 60 | 75 | | | |
| | 1.8 | 9 | 10.8 | 14.4 | 18 | 27 | 36 | 45 | 54 | 63 | 72 | 90 | | | |
| | 2.0 | 10 | 12 | 16 | 20 | 30 | 40 | 50 | 60 | 70 | 80 | 100 | | | |
| | 2.5 | 12.5 | 15 | 20 | 25 | 37.5 | 50 | 62.5 | 75 | 87.5 | 100 | | | | |
| | 3.0 | 15 | 18 | 24 | 30 | 45 | 60 | 75 | 90 | | | | | | |
| | 3.5 | 17.5 | 21 | 28 | 35 | 52.5 | 70 | 87.5 | | | | | | | |

As shown in Table 1.1, the highlighted horizontal axis indicates the overall repetition rates of the output laser pulses generated by one or more laser cavities 141A-D, and the highlighted vertical axis indicates the pulse energy levels of output laser pulses generated by the one or more laser cavities 141A-D. An average power output level of a laser pulse may be obtained by inputting, for example, via the user interface 104, a repetition frequency, and a pulse energy level indicated in a spectrum matrix (e.g., Table 1.1). For example, in order to generate a laser pulse having an average output of 4 Watts (W), a user may input, via the user interface 104, a repetition frequency of 8 Hz and a pulse energy level of 0.5 Joules (J). In one example, in order to generate an output laser pulse having an overall repetition frequency below 10 Hz (e.g., 5 Hz, 6 Hz, 8 Hz, etc.), the controller 110 may automatically generate one or more signals to control a single laser cavity (e.g., any one of the four cavities) to generate the output laser pulse. Additionally or alternatively, controller 110 may control: two laser cavities to generate an output laser pulse having an overall repetition frequency at 10 Hz to 14 Hz; three or more laser cavities to generate output laser pulses having overall repetition frequencies of 15 Hz to 19 Hz; and four laser cavities to generate output laser pulses having overall repetition frequencies at 20 Hz or higher. Of course, the spectrum matrix may be varied based on the operating capabilities of the medical laser system 100. Further, additional spectrum matrices may be programmed or generated based on different laser applications and/or treatments.

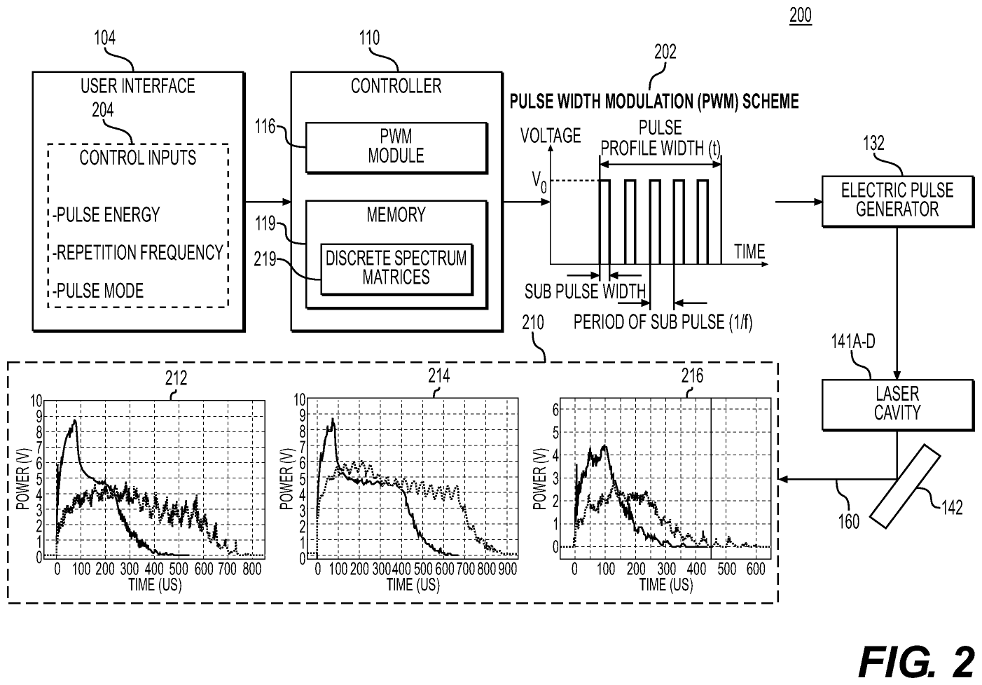

FIG. 2 shows an exemplary laser pulse generation process 200 that utilizes PWM scheme techniques to generate laser pulses having one or more laser pulse modes (or shapes) in accordance with one or more aspects of this disclosure. In one exemplary embodiment of process 200, the user interface 104 may receive control inputs 204 from a user (or an operator). The control inputs 204 may include, for example, pulse energy data (or value), repetition frequency data (or value), and/or pulse mode data (or value) associated with the output laser pulse 160. The pulse energy data and the repetition frequency data may correspond to, for example, one or more parameters listed in one or more discrete spectrum matrices 219 (e.g., PRF matrix is shown in Table 1.1) stored in the memory 119. The laser pulse mode data may correspond to one or more laser pulse shapes that may be generated by the medical laser system 100 of this disclosure. For example, one or more laser pulse modes may include a regular pulse, a short pulse, a long pulse, a very long pulse, a dust pulse, and a burst pulse. The PWM module 116 may generate PWM control signals to modulate electric pulse signals in order to generate laser pulses having various modes (or shapes). In one embodiment, one or more parameters associated with one or more laser pulse modes may be programmed or stored in memory 119 in order to integrate the parameters of one or more pulse modes with an existing spectrum matrix (e.g., PRF matrix).

Still referring to FIG. 2, the PWM module 116 may facilitate a laser pulse shaping technique to optimize the effects of interaction between the output laser pulses and target substances (e.g., tissues). In one embodiment, the PWM module 116 may utilize at least three parameters for a set voltage of the power supply (e.g., a capacitor bank) of the system 100, to define the features of an electrical pumping pulse profile. At least three parameters may include a sub-pulse period, a pulse profile width, and a discharging sub-pulse width. Based on at least the three parameters, the PWM module may derive an equivalent set of parameters: a sub-pulse frequency (f) of a PWM pulse signal, a pulse profile width (t) of the PWM control signal, and an overall electric pulse width (τ). The relationship between the two sets of parameters may be derived, for example, in accordance with the following algorithm:

$$N = t \cdot f$$

$$T = \frac{1}{f}$$

$$Δτ = \frac{τ}{N} = \frac{τ}{t \cdot f}$$

$$ρ = \frac{τ}{t}$$

The number of sub-pulses contained in a pulse profile of a PWM control signal may be defined as N, the period of a sub-pulse of the PWM control signal may be defined as T, the width of the sub-pulse of the PWM control signal may be defined as Δτ, and the duty cycle (actual electric pulse width (τ) versus the PWM control pulse profile width (t)) may be defined as ρ.

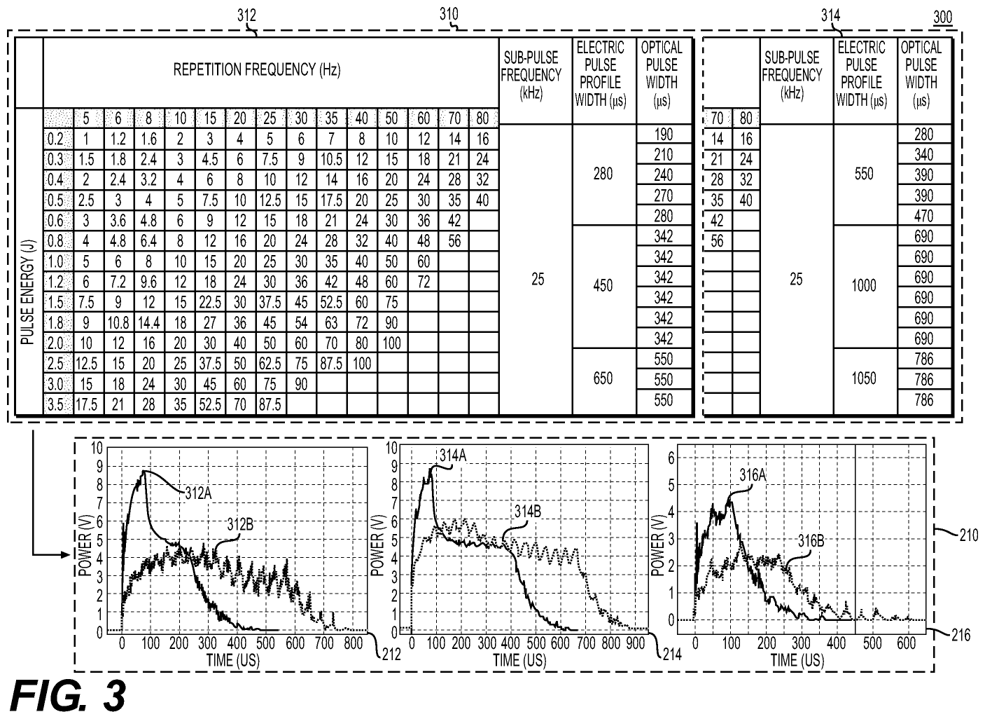

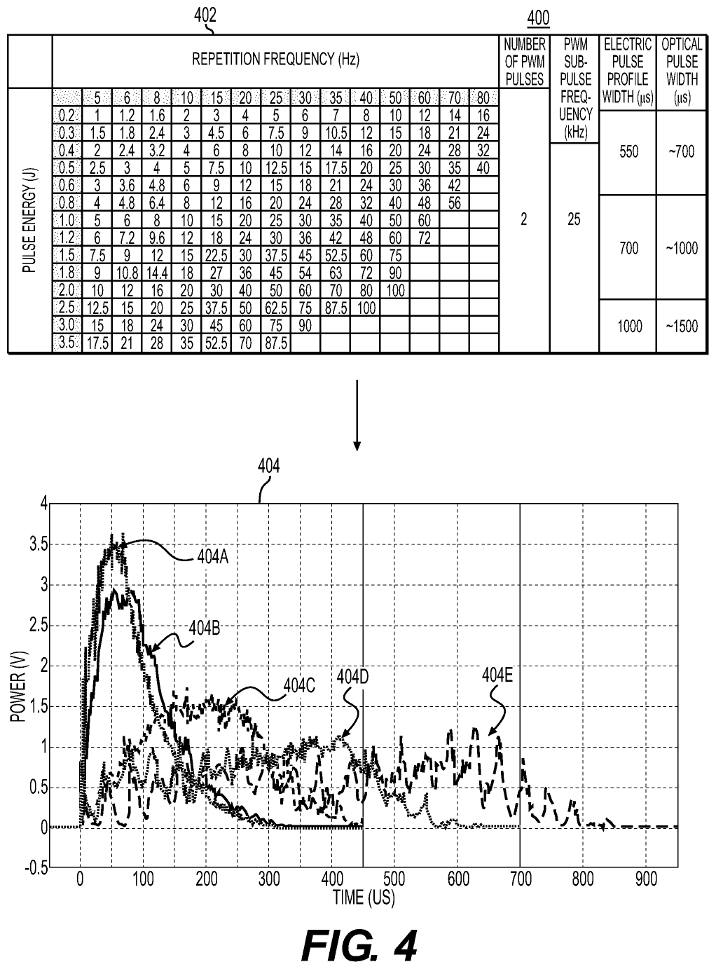

In one embodiment, a PWM control signal may be defined with a given sub-pulse frequency (f) and a pulse profile width (t). Further, the actual laser (or optical) pulse energy may be varied by adjusting the overall electric pulse width (τ). That is, adjusting the overall electric pulse width (τ) may change the duty cycle (ρ), which may, in turn, cause an overall laser pulse energy to change, with little or no change in the pulse profile width (t). With the PWM scheme technique of this disclosure, the pulse shapes or modes (e.g., a regular pulse, a short pulse, a long pulse, a very long pulse, a dust pulse, a burst pulse, etc.) of a laser pulse may be achieved and manipulated. Since the sub-pulse frequency (f) and pulse profile width (t) of a PWM pulse signal may determine the type (e.g., mode or shape) of a laser pulse, these parameters may be specified and defined in the one or more discrete spectrum matrices 219. For example, a laser pulse generated by a sub-pulse frequency (f) specified in the range of 10 kHz to 15 kHz (e.g., 12 kHz) may be defined as a dust pulse, and by a sub-pulse frequency (f) specified in the range of 3 kHz to 5 kHz may be defined as a burst pulse. Additionally, a short pulse, a long pulse, and a very long pulse may be further defined based on one or more parameters of the one or more discrete spectrum matrices 219. (Later shown in 312, 314 of FIG. 3 and 402 of FIG. 4)

Still referring to FIG. 2, upon receiving the control inputs 204 from the user interface 104, the PWM module 116 may communicate with the memory 119 to access or receive information from the one or more discrete spectrum matrices 219. The PWM module 116 may then generate one or more PWM control signals 202 based at least on the received control inputs 204 (e.g., pulse energy data, repetition frequency data, laser pulse mode data, etc.) and the one or more discrete spectrum matrices 219. The PWM module 116 may then transmit the PWM pulse signals 202 to the electric pulse generator 132. The electric pulse generator 132 may generate modulated electric pulse signals based on the received PWM pulse signals 202. The electric pulse generator 132 may then transmit the modulated electric signals to one or more laser cavities 141A-D. The one or more laser cavities 141A-D may then generate laser (or optical) pulses based on the modulated electric signals received from the electric pulse generator 132 and transmit the generated laser pulses to the rotating mirror 142 to generate an output laser pulse 160.

Still referring to FIG. 2, the generated output laser pulse 160 may comprise one or more pulse modes 210. For example, the one or more pulse modes 210 may be: a short or long pulse with high pulse energy 214 (e.g., approximately 3500 mJ); a short or long pulse with medium pulse energy 212 (e.g., approximately 2000 mJ); and a short or pulse with low pulse energy 216 (e.g., approximate 600 mJ). The PWM technique of this disclosure overcomes the difficulty of specifying individual sub-pulses for obtaining a desired laser pulse mode. For example, a sub-pulse frequency (f) and a pulse profile width (t) of a PWM control signal may be predefined for all modes of laser pulses. Thereafter, the overall electric pulse width (τ) may be adjusted by a user or operator to obtain a desired laser pulse mode. Additionally, laser pulses having different pulse energy levels may be achieved by changing the pulse width (τ) parameter. As discussed above, laser pulses with different pulse energy levels may have the same frequency (f) and approximately the same pulse width (t). That is, the pulse energy may be adjusted based on the change in the sub-pulse duty cycle (ρ) of a PWM control signal.

FIG. 3 shows an exemplary process 300 for generating laser pulses having one or more pulse modes (or shapes) by utilizing discrete spectrum matrices 310 in accordance with the system and process disclosed in FIGS. 1 and 2. The discrete spectrum matrices 310 may include a short pulse matrix 312 and a long pulse matrix 314. In one embodiment, the short pulse matrix 312 and the long pulse matrix 314 may include pulse energy and repetition frequency parameters identical to the parameters included in the PRF matrix shown in Table 1.1. In addition, the short pulse matrix 312 and the long pulse matrix 314 may include pulse mode parameters for generating short laser pulses 312A-316A and long laser pulses 312B-316B, respectively. For example, the pulse mode parameters for the short pulse matrix 312 may include a sub-pulse frequency of 25 kHz and electric pulse profile widths ranging approximately between 280 μs to 650 μs. The pulse mode parameters for the long pulse matrix 314 may include a sub-pulse frequency of 25 kHz and electric pulse profile widths ranging approximately between 550 μs to 1050 μs. In one embodiment, a short laser pulse may be defined as a laser pulse that may be generated based on the parameters available on the short pulse matrix 312. For example, short laser pulses generated based on the short pulse matrix 312 may include: the short laser pulse 314A generated with a pulse energy of 3500 mJ (high energy 314) having a pulse profile width of approximately 650 μs; the short laser pulse 312A generated with a pulse energy of 2000 mJ (medium energy 312) having a pulse profile width of approximately 500 μs; and the short laser pulse 316A generated with a pulse energy of 600 mJ (low energy 316) having a pulse profile width of approximately 350 μs. Further, a long laser pulse may be defined as a laser pulse that is generated based on the parameters available on the long pulse matrix 314. For example, long laser pulses generated based on the long pulse matrix 314 may include: the long laser pulse 314B generated with a pulse energy of 3500 mJ (high energy 314) having a pulse profile width of approximately 900 μs; the long laser pulse 312B generated with a pulse energy of 2000 mJ (medium energy 312) having a pulse profile width of approximately 800 μs; and the long laser pulse 316B generated with a pulse energy of 600 mJ (low energy 316) having a pulse profile width of approximately 550 μs. In this example, any pulse having a pulse profile width that is more than 50% longer than a pulse profile width of a short laser pulse generated at the same pulse may be defined as a long laser pulse. Furthermore, a very long laser pulse may be defined as a laser pulse that is generated based on the parameters available on an expanded spectrum matrix (later shown in FIG. 4).

In one exemplary process, a user may input (or select), via the user interface 104, a high pulse energy level (e.g., 3500 mJ). Based on the short-pulse matrix 312 or the long spectrum matrix 314, the user may then select a repetition frequency between any one of 7.5, 21, 28, 35, 52.5, 70, or 87.5 Hz. Additionally, the user may input or select a laser pulse mode (e.g., a short pulse or a long pulse). The PWM module 116 may then automatically generate either a short laser pulse 314A having an optical pulse width between 190 and 550 μs or a long laser pulse 314B having an optical pulse width between 280 and 786, in accordance with the short pulse matrix 312 and the long pulse matrix 314. As shown in the short and long pulse matrices 312 and 314, the optical pulse width of the short laser pulse 314A or the long laser pulse 314B may vary depending on the selected pulse energy, repetition frequency, sub-pulse frequency, and the electric pulse profile width.

In one example, a user may input or select, via the user interface 104, each parameter of the discrete spectrum matrices 310, individually or separately, in order to generate a desired laser pulse mode (or shape). Alternatively, each laser pulse mode may be predefined (or preprogrammed) based on pre-selected parameters. That is, a set of parameters (e.g., pulse energy of 3.5 J; repetition frequency of 4 Hz; sub-pulse frequency of 25 kHz; and electric pulse profile width of 650 μs) may be selected, for example, as a selectable graphical or text item on the user interface 104, based on a predefined specific laser pulse mode (e.g., “High energy, a short pulse having an optical pulse width of 550 μs”). The short pulse matrix 312 and the long pulse matrix 314 are shown in FIG. 3 include exemplary combinations of parameters. That is, various different combinations of parameters may be selected, in accordance with one or more aspects of this disclosure, for generating different discrete spectrum matrices to define one or more desired laser pulse modes.

In some examples, the medical laser system 100 of this disclosure may cover large ranges of parameters, such as, for example, pulse energy, repetition frequency, and laser pulse modes. As such, the ranges of parameters may be divided into smaller groups of laser pulse modes. That is, each laser pulse mode may contain pulses of similar features and parameters in a common range. For example, a dust pulse or a burst pulse may each belong to an independent pulse mode, as all dust or burst pulses may include the same PWM frequency. Further, a regular pulse may be separated into different pulse modes according to pulse energy. In one embodiment, laser pulses with pulse energy levels less than 600 mJ may be grouped into “Small Pulses” mode; pulse energy levels between 800 mJ to 2000 mJ may be grouped into “Medium Pulses” mode, and pulse energy levels higher than 2000 mJ into “Large Pulses” mode. Since at least three different types of pulse widths may be available for each pulse energy and repetition rate (i.e., short, long, and very long pulse), there may be two or more small, medium, and/or large laser pulse modes.

In some embodiments, one or more specific pulse modes may be programmed and stored in the memory 119 (or a database), in the form of one or more multi-dimensional (e.g., 3-dimensional) tables that may be accessible by the calibration module 114, the PWM module 116, and/or the monitoring and adjustment module 118. For example, the one or more multi-dimensional tables may define one or more laser pulse modes including, but not limited to: a small (energy) short pulse, a small long pulse, a medium-short pulse, a medium-long pulse, a large short pulse, a large long pulse, small very long pulse, medium very long pulse, large very long pulse, a dust pulse, a burst pulse, etc. As such, the one or more multi-dimensional tables may include predefined selectable parameters (e.g., pulse energy, repetition frequency, sub-pulse frequency, electric pulse profile width, etc.) for each of the one or more laser pulse modes.

FIG. 4 shows another exemplary process 400 for generating laser pulses having one or more pulse modes (or shapes) by utilizing an expanded pulse matrix 402 in accordance with the system and processes disclosed in FIGS. 1-3. In one exemplary embodiment of the process, the expanded pulse matrix 402 may comprise one or more parameters for generating laser pulses with a longer optical pulse width than the short and long pulses, as described in accordance with FIG. 3 above. That is, an optical pulse width of a laser pulse may be increased by more than 2 times the optical pulse width of the short laser pulse generated in accordance with process 300 of FIG. 3. In one embodiment, a very long laser pulse may be defined as a laser pulse comprising an optical pulse width that is greater than 2 times the optical pulse width generated by the short pulse matrix 312.

Still referring to FIG. 4, the expanded pulse matrix 402 may include pulse energy and repetition frequency parameters identical to the parameters included in the PRF matrix shown in Table 1.1. In addition, the expanded pulse matrix 402 may include laser pulse mode parameters for generating laser pulses 404 with various pulse modes. In this embodiment, the laser pulse mode parameters may include, for example, the number of PWM pulses, PWM sub-pulse frequency, and electric pulse profile width. With the addition of specifying the number of PWM pulses in the expanded pulse matrix 402, a variety of laser pulse modes may be achieved. For example, a regular single pulse 404A may be generated in accordance with the system and process disclosed in FIGS. 1-4. Additionally or alternatively, laser pulses having different modes 404B-404E may be generated by adjusting the number of PWM pulses utilized in the laser pulse mode parameters in the expanded pulse matrix 402. For example, in order to generate laser pulses 404B, 404C, 404D, and 404E, a single PWM pulse combined two PWM pulses, combined three PWM pulses, and combined four PWM pulses may be utilized, respectively. For example, laser pulses generated based on the PWM pulse matrix 402 with the overall pulse energy of approximately 300 mJ may include: the regular single pulse 404A having a pulse profile width of approximately 350 μs; the single PWM pulse 404B having a pulse profile width of approximately 350 μs; the combined two PWM pulse 404C generated having a pulse profile width of approximately 450 μs; the combined three PWM pulse 404D generated having a pulse profile width of approximately 600 μs, and the combined four PWM pulse 404E generated having a pulse profile width of approximately 850 μs. In this example, any pulse having a pulse profile width greater than 2 times the width of a regular single pulse generated at the same pulse may be defined as a very long laser pulse.

In one embodiment, the laser pulses utilizing the expanded pulse matrix 402 may be generated in a similar manner as disclosed in accordance with process 300 for generating the short and long laser pulses. Additionally or alternatively, a special code may be entered in the user interface 104 to indicate that a preset number of PWM pulses may be utilized or combined for generating laser pulses in accordance with the expanded pulse matrix 402.

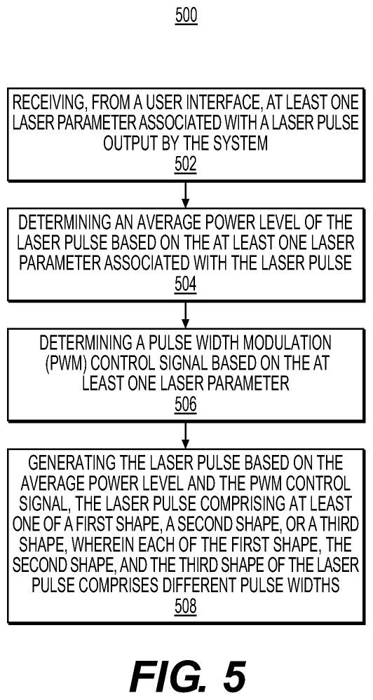

FIG. 5 shows an exemplary process 500 for generating laser pulses having one or more pulse modes (or shapes) by utilizing multiple laser cavities and PWM scheme techniques in accordance with the system and processes disclosed in FIGS. 1-4. This exemplary process may allow the shaping of laser pulses, which may improve the effects of performing laser applications or treatments on target features, such as tissues of a subject (e.g., patient).

At step 502, system 100 may receive, from a user interface (e.g., user interface 104), at least one laser parameter data associated with a laser pulse output by the system. In one embodiment, at least one parameter associated with the laser pulse may include at least pulse mode data, pulse repetition frequency data, or pulse energy data. In one embodiment, the shape of the laser pulse may be determined based at least on the pulse mode data. At step 504, controller 110 may determine an average power level of the laser pulse based on at least one laser parameter associated with the laser pulse. In one embodiment, the average power level of the laser pulse may be determined based on a discrete spectrum matrix. The discrete spectrum matrix may comprise parameters based at least on the pulse energy data of the laser pulse, the repetition frequency data of the laser pulse, sub-pulse frequency of a PWM pulse, and pulse profile width data of the PWM pulse, and overall pulse width data of the laser pulse. In one embodiment, one or more discrete spectrum matrices comprising parameters specifying one or more pulse modes of the laser pulse may be stored in the memory 119.

At step 506, the PWM module 116 may determine a pulse width modulation (PWM) control signal based on the pulse mode data. At step 508, the laser assembly 140 (e.g., one or more laser cavities 141A-D) may generate the laser pulse based on the average power level and the PWM control signal. The laser pulse may comprise at least one of a first shape, a second shape, or a third shape, and each of the first shape, the second shape, and the third shape of the laser pulse comprises different pulse widths (e.g., short, long, or very long laser pulse). In one embodiment, a pulse width of the second shape may be at least 50% greater than the pulse width of the first shape. In one embodiment, the pulse width of the third shape of the laser pulse may be at least 2 times greater than the pulse width of the first shape of the laser pulse. The third shape of the laser pulse may be generated by combining multiple PWM control signals. In one embodiment, the laser assembly 140 may generate a laser pulse having a repetition frequency of 10 Hertz (Hz) or greater by combining multiple laser pulses generated by at least two optical channels (e.g., laser cavities 141A-D). The multiple laser pulses may be combined by synchronizing a rotating mirror with the multiple laser pulses generated by at least two optical channels.

The medical laser system 100 of this disclosure allows a user to shape (or adjust) the pulse profiles of laser pulses by modulating the laser pulses in accordance with the PWM scheme techniques disclosed above. The modulated laser pulses using the PWM scheme techniques may generate various laser pulses, including, but not limited to, regular, short, long, very long, dust, and burst pulses. As such, the number of working points (or laser pulse modes) for the medical laser system 100 may be greatly expanded (e.g., with different combinations of pulse energy, repetition rate, pulse profile shapes). Accordingly, the medical laser system 100 of this disclosure may generate laser pulses that may be utilized in a variety of medical applications.

It will be understood that reference is made to a number of cavities and/or mirrors in the medical laser system 100. It will be understood that the devices are not limited to this number and may change according to the requirement of the medical laser system 100. Further, while reference is made to a medical/surgical laser system, the laser pulse technique described herein is not limited to a medical/surgical laser system and may be used with any laser system.

It will be apparent to those skilled in the art that various modifications and variations may be made in the disclosed systems and methods without departing from the scope of the disclosure. It should be appreciated that the disclosed system may include various suitable computer systems and/or computing units incorporating a plurality of hardware components, such as, for example, a processor and non-transitory computer-readable medium, that allow the devices to perform one or more operations during a procedure in accordance with those described herein. Other aspects of the disclosure will be apparent to those skilled in the art from consideration of the specification and practice of the features disclosed herein. It is intended that the specification and examples be considered exemplary only.

It should be appreciated that the controller 110 in FIG. 1 may be any computing device. The user interface 104 also may include input and output ports to connect with input and output devices such as keyboards, mice, touchscreens, monitors, displays, etc. Of course, the various system functions may be implemented in a distributed fashion on a number of similar platforms to distribute the processing load. Alternatively, the systems may be implemented by appropriate programming of one computer hardware platform.

In one embodiment, any of the disclosed systems, methods, and/or graphical user interfaces may be executed by or implemented by a computing system consistent with or similar to the descriptions herein. Although not required, aspects of this disclosure are described in the context of computer-executable instructions, such as routines executed by a data processing device, e.g., a server computer, wireless device, and/or personal computer. Those skilled in the relevant art will appreciate that aspects of this disclosure can be practiced with other communications, data processing, or computer system configurations, including Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “computing device,” and the like are generally used interchangeably herein and refer to any of the above devices and systems, as well as any data processor.

Aspects of this disclosure may be embodied in a special purpose computer and/or data processor that is specifically programmed, configured, and/or constructed to perform one or more of the computer-executable instructions explained in detail herein. While aspects of this disclosure, such as certain functions, are described as being performed exclusively on a single device, this disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein involving multiple devices may be implemented in a single device. In a distributed computing environment, program modules may be located in both local and/or remote memory storage devices.

Aspects of this disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer-implemented instructions, data structures, screen displays, and other data under aspects of this disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet-switched, circuit-switched, or another scheme).

Program aspects of the technology may be thought of as “products” or “articles of manufacture,” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives, and the like, which may provide non-transitory storage at any time for the software programming. All or portions of the software may at times be communicated through the Internet or various other telecommunication networks. Such communications, for example, may enable the loading of the software from one computer or processor into another, for example, from a management server or host computer of the mobile communication network into the computer platform of a server and/or from a server to the mobile device. Thus, another type of media that may bear the software elements includes optical, electrical, and electromagnetic waves, such as used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

It will be apparent to those skilled in the art that various modifications and variations can be made to the disclosed system without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical laser system for outputting laser pulses, the system comprising:
at least one laser cavity;
a rotating mirror;
a user interface; and
a controller configured to:
receive, from the user interface, at least one laser parameter associated with a laser pulse output by the system;
determine an average power level of the laser pulse based on the at least one laser parameter associated with the laser pulse;
determine a pulse width modulation (PWM) control signal based on at least one laser parameter; and
generate the laser pulse based on the average power level and the PWM control signal, the laser pulse comprising at least one of a first shape, a second shape, or a third shape,
wherein each of the first shape, the second shape, and the third shape of the laser pulse comprises different pulse widths.

2. The system of claim 1, wherein the at least one laser parameter comprises at least pulse mode data, pulse repetition frequency data, or pulse energy data associated with the laser pulse.

3. The system of claim 1, wherein a pulse width of the second shape is at least 50% greater than a pulse width of the first shape.

4. The system of claim 1, wherein a pulse width of the third shape of the laser pulse is at least 2 times greater than a pulse width of the first shape of the laser pulse.

5. The system of claim 1, wherein a shape of the laser pulse is determined based at least on pulse mode data.

6. The system of claim 1, wherein the average power level of the laser pulse is determined based on a discrete spectrum matrix, and
wherein the discrete spectrum matrix comprises parameters based at least on pulse energy data of the laser pulse, repetition frequency data of the laser pulse, sub-pulse frequency of a PWM pulse, pulse profile width data of the PWM pulse, or overall pulse width data of the laser pulse.

7. The system of claim 1, wherein the one or more optical channels comprises a holmium-doped yttrium aluminum garnet (Ho:YAG) rod.

8. The system of claim 1, wherein the at least one laser cavity comprises four laser cavities.

9. The system of claim 1, wherein the at least one laser cavity comprises at least two laser cavities; and
wherein the controller is further configured to:
generate a laser pulse having a repetition frequency of 10 Hertz (Hz) or greater by combining multiple laser pulses generated by the at least two laser cavities,
wherein the multiple laser pulses are combined by synchronizing the rotating mirror with the multiple laser pulses generated by the at least laser cavities.

10. The system of claim 1, wherein the controller is further configured to:
generate the laser pulse having the third shape by combining multiple PWM control signals.

11. The system of claim 1, wherein the controller is further configured to:
store one or more discrete spectrum matrices comprising parameters specifying one or more pulse modes of the laser pulse.

12. A method of outputting laser pulses of a medical laser system, the method comprising:
receiving, from a user interface, at least one laser parameter associated with a laser pulse output by the system;
determining an average power level of the laser pulse based on the at least one laser parameter associated with the laser pulse;
determining a pulse width modulation (PWM) control signal based on the at least one laser parameter; and
generating the laser pulse based on the average power level and the PWM control signal, the laser pulse comprising at least one of a first shape, a second shape, or a third shape,
wherein each of the first shape, the second shape, and the third shape of the laser pulse comprises different pulse widths.

13. The method of claim 12, wherein the at least one laser parameter comprises at least pulse mode data, pulse repetition frequency data, or pulse energy data associated with the laser pulse.

14. The method of claim 12, wherein a pulse width of the second shape is at least 50% greater than a pulse width of the first shape; and
wherein a pulse width of the third shape of the laser pulse is at least 2 times greater than a pulse width of the first shape of the laser pulse.

15. The method of claim 12, wherein a shape of the laser pulse is determined based at least on pulse mode data.

16. The method of claim 12, wherein the average power level of the laser pulse is determined based on a discrete spectrum matrix, and
wherein the discrete spectrum matrix comprises parameters based at least on pulse energy data of the laser pulse, repetition frequency data of the laser pulse, sub-pulse frequency of a PWM pulse, pulse profile width data of the PWM pulse, or overall pulse width data of the laser pulse.

17. The method of claim 12 further comprising:
generating a laser pulse having a repetition frequency of 10 Hertz (Hz) or greater by combining multiple laser pulses generated by at least two laser cavities,
wherein the multiple laser pulses are combined by synchronizing a rotating mirror with the multiple laser pulses generated by the at least two laser cavities.

18. The method of claim 12 further comprising:
generating the laser pulse having the third shape by combining multiple PWM control signals.

19. The method of claim 12 further comprising:
storing one or more discrete spectrum matrices comprising parameters specifying one or more pulse modes of the laser pulse.

20. A non-transitory computer-readable medium storing instructions for outputting laser pulses of a medical laser system, the instructions, when executed by one or more processors, causing the one or more processors to perform operations comprising:
receiving, from a user interface, at least one laser parameter associated with a laser pulse output by the system;
determining an average power level of the laser pulse based on the at least one laser parameter associated with the laser pulse;
determining a pulse width modulation (PWM) control signal based on the at least one laser parameter; and
generating the laser pulse based on the average power level and the PWM control signal, the laser pulse comprising at least one of a first shape, a second shape, or a third shape,
wherein each of the first shape, the second shape, and the third shape of the laser pulse comprises different pulse widths.
